(19) United States
(12) Patent Application Publication
(10) Pub. No.: US 2010/0242970 A1
(43) Pub. Date: Sep. 30, 2010

(54) System for mandibular protrusion to prevent snoring and apnea

(76) Inventor: JUERGEN SCHMITT-BYLANDT, GELNHAUSEN (DE)

Correspondence Address: COLLARD & ROE, P.C., 1077 NORTHERN BOULEVARD, ROSLYN, NY, 11576, US

(21) Appl. No.: 12/761,626

(22) Filed: Apr. 16, 2010

Related U.S. Application Data
(63) Continuation-in-part of application No. PCT/DE2008/001683, filed on Oct. 19, 2008.

(30) Foreign Application Priority Data
Oct. 19, 2007 (DE) 10 2007 050 309.3
Oct. 14, 2008 (DE) 10 2008 051 221.4

(51) Int. Cl. A61F 5/56

(52) U.S. Cl. 128/848

(57) ABSTRACT

A system for preventing snoring and apnea prevents the mandible from dropping back when sleeping. Contrary to familiar methods, the system is inconspicuous and is highly comfortable in use, which are prerequisites for the compliance of patients and lasting use. For this purpose, in an expanded embodiment, the splints are connected to the resilient clamps or brackets using a hinge mechanism, which allows a free rotational movement of the jaw joints corresponding to the Bennett angle.

### CROSS REFERENCE TO RELATED APPLICATIONS

Applicant claims priority on and this application is a continuation-in-part under 35 U.S.C. 120 of International Application No. PCT/DE2008/001683 filed Oct. 19, 2008, which claims priority under 35 U.S.C. 119 of German Application No. 10 2007 050 309.3 filed Oct. 19, 2007 and German Application No. 10 2008 051 221.4 filed Oct. 14, 2008. The International Application under PCT Article 21(2) was not published in English. Applicant also claims priority under 35 U.S.C. 119 of German Application No. 10 2007 050 309.3 filed Oct. 19, 2007 and German Application No. 10 2008 051 221.4 filed Oct. 14, 2008. The disclosure of the aforesaid International Application and German applications are incorporated by reference.

### BACKGROUND OF THE INVENTION

Problems through snoring when living together and in partnerships are widely known and the conditional restrictions and health risks of temporary cessation of breathing (apnea) caused thereby are well documented medically [1 to 4]. Numerous attempts have therefore been made to prevent snoring technically or by influencing behaviour.

However, only operative intervention to shorten the soft palate and the use of so-called protrusion splints, which prevent the mandible from falling back during sleep, have proved to be generally effective hitherto.

Whilst an operative intervention appears to be justified only in serious cases, the protrusion splints used hitherto entail serious drawbacks in use, which prevent a long-term use even after the best possible individual adjustment.

The majority of the methods known hitherto operate here with in each case a splint placed onto the row of teeth of the maxilla and mandible, which are connected with each other via adjustable joints so that the mandible can not fall back during sleep when the masseters relax.

This is, in fact, entirely effective with regard to preventing snoring. However, it is difficult to adapt the joint mechanism so that natural movements of the mandible are not prevented, in particular that no tensioning of the temperomandibular joints and cramps of the chewing musculature occur. However, even with optimum adjustment, the desired restriction to the motivity of the jawbones is inevitably felt to be adverse. Thus, for example, yawning is only possible to a limited extent, speaking is made extremely difficult and the forced positioning of the jawbones is intrusive.

The splints themselves, which are placed onto the rows of teeth of the jawbones, are additionally highly intrusive, which are similar in construction to dental prostheses, but generally prevent the habitual closing of the dentition through the fact that they overlap the masticatory surfaces. In addition, the necessity of a largely fixed connection with the rows of teeth of both jawbones involves the splints coming in contact with freely standing necks of teeth and with the gums and causing irritations and occasional inflammations there.

Nevertheless, numerous attempts are known to overcome these disadvantages, which, however, have only been partially effective hitherto, and therefore virtually restrict the use of the protrusion splints to extreme cases in which the drawbacks must be accepted as being unavoidable:

Thus with methods which provide a largely fixed connection between the maxilla and mandible (FR 27 27 008, JP 2004 073 473), the least compliance of the patients is achieved, systems such as clamps making partial movements possible according to JP 2006 095 245, which restrict the tongue movement and are to be clamped in a similar manner to dental prostheses in tooth crowns have just as little acceptance.

With the systems of Schlieper (DE 100 29 875, DE 102 16 242 and DE 103 31 531) an attempt is made to at least keep the tongue cavity free, in order to permit undisturbed speech to some extent. Whereas the former still describes a soft elastic connection and hence very restricted movement of the two splints with respect to each other, the subsequent developments are equipped with telescope-like adjustable (but then rigid) connections (so-called Herner telescope) which the earlier widely-used protrusion splints according to Hinz also had (inter alia marketed by Scheu-Dental, Esslingen) and involve the disadvantages described above.

U.S. Pat. No. 4,901,737 is likewise intended to keep the tongue cavity free, however provides a prosthesis-like insert on the mandible side, which is equipped with a metal clip for the maxilla which is evidently complicated to insert (and expensive to produce individually).

In addition, suggestions which prevent a falling back of the tongue by clamping (JP 2005 312 853 and US 2006/130850, or by holding forward with underpressure (FR 2 769 496) have not proved to be successful, evidently due to lack of acceptance by the patients.

Systems which are intended to adjust the jawbones by supporting in the palate cavity (U.S. Pat. No. 5,117,816) or to restrict them by cam inserts between the rows of teeth (U.S. Pat. No. 5,003,994, DE 10 2004 007 008), or are in fact adjustable on insertion but then rigid (such as U.S. Pat. No. 5,570,704), or respectively are only movable in longitudinal direction as in DE 10 2004 058 081, are likewise problematic, because this respectively is contrary to a natural jawbone movement.

On the other hand, systems in which the forward push of the mandible with respect to the maxilla takes place in an elastic manner, for instance by the insertion of rubber bands, as described in CA 1998 223 65 03 and U.S. Pat. Nos. 5,947,724/5,794,627, or in U.S. Pat. No. 5,570,704 are better accepted. Here, however, relatively solid splints on the rows of teeth and projecting mountings into which the patient must hook the rubber bands are intrusive.

In addition, in almost all cases clips are provided which enclose the rows of teeth on three sides. However, except for the above-mentioned solutions, they prevent the movement of the tongue and are therefore highly intrusive when speaking. In each case, however, they are felt as a foreign body in the mouth cavity and all the more so, the thicker they are and the more they exert pressure onto sensitive areas, e.g. the gums.

On the other hand, designs which in fact protect the gums by correspondingly soft cushioning, such as U.S. Pat. No. 5,003,994, U.S. Pat. No. 5,829,441 and EP 1 203 570, but to do this are applied relatively thickly, are scarcely able to be tolerated in the long term, because the patient can not close his mouth with it. In addition, a forced opening between the jawbones for the entry of air, which patients feel to be intrusive, is medically controversial or only really necessary in cases of severe apnea.

### SUMMARY OF THE INVENTION

It is therefore an object of the present invention to find a solution which prevents a falling back of the mandible during sleep, but which in so doing only stresses the patient minimally. He should be able to speak and yawn unimpeded, and be able to move the jawbone in all usual directions, the splints should be scarcely intrusive in the mouth and should also be as invisible as possible, and the protrusion of the mandible should take place elastically and only be so intensive that a quasi-natural jawbone position is achieved and felt.

This is solved according to the invention in that splints, e.g. of PET, are used which—preferably by deep-drawing—are shaped so that the bearing on the tooth surfaces is thin and on the flanks is only of necessary intensity, the front tongue cavity and the tooth necks and the gums remain open and only an elastic connection of the mandible and maxilla brings about the protrusion.

For this, the metal clamps are arranged at rear ends of the splints or in other words the splints, which are preferably transparent, are connected with two clamps of elastic material, preferably a titanium-nickel memory metal connection, which can be pre-produced in suitable embodiments and given a fine adjustment if necessary with screw inserts, or can be fixed after individual fitting by heating.

Depending on the jawbone and joint position, these clamps can be embodied as comparatively short curves or if necessary can have an elastic loop.

In the case of unfavourable tooth positions or tooth shapes, which prevent a secure seat of the protrusion splints (e.g. in the case of so-called pyramid teeth), it is, in addition, possible to supplement the splints with mechanisms such as e.g. hooks and clips, which are known from tooth or jawbone clips, in order to improve the hold in the rows of teeth.

In an extended embodiment of this system (Prior Application of Oct. 14, 2008) it is proposed to follow further the natural kinematics of the mandible by corresponding further to the change in the Bennett angle, which is produced from the lateral and rotational movement of the mandible joints, in that the protrusion is uncoupled through the pre-stressing between mandible and maxilla by the rotation of the temperomandibular joint through inclusion of a further joint, which—arranged almost parallel to the temperomandibular joint—gives the connection an additional degree of freedom in the planes of rotation.

Through this arrangement, in which the splint system itself becomes a joint, it is ensured that despite a defined protrusion of the mandible, the physiological stressing of the band apparatus and the chewing musculature remains low.

In addition, also with further opening of the mouth, e.g. when yawning, no withdrawing forces are exerted onto the splints.

They can therefore be embodied so as to be thinner and do not have to be pressed under tension onto the rows of teeth, which otherwise considerably impedes their removal again.

### BRIEF DESCRIPTION OF THE DRAWINGS

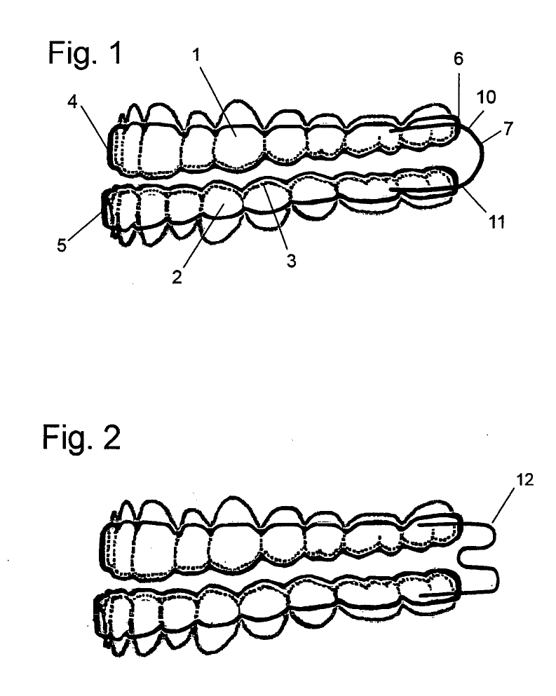

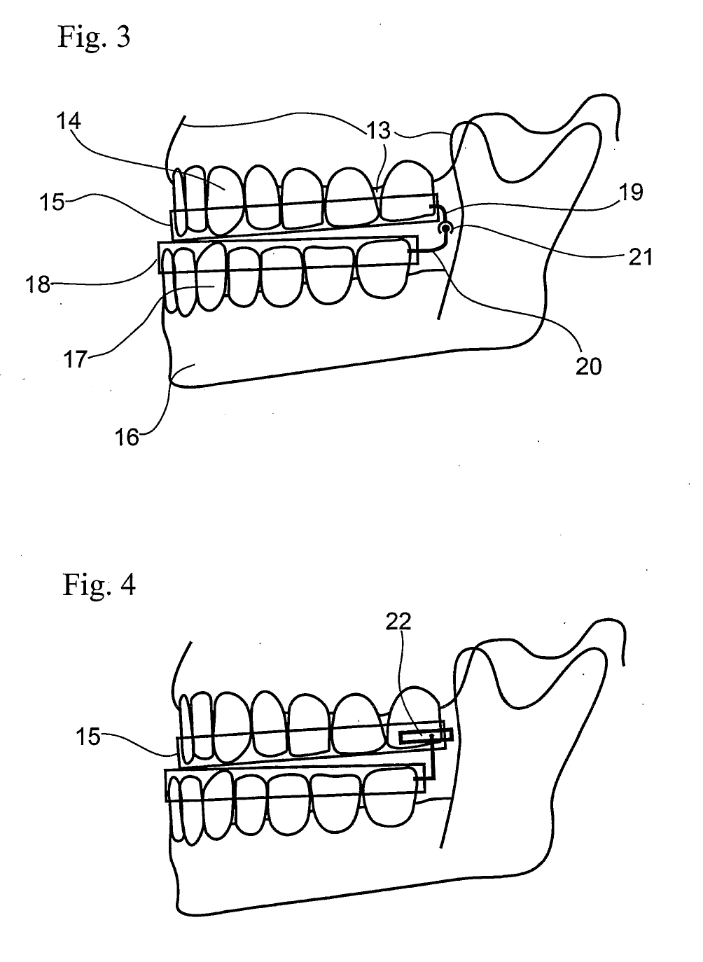

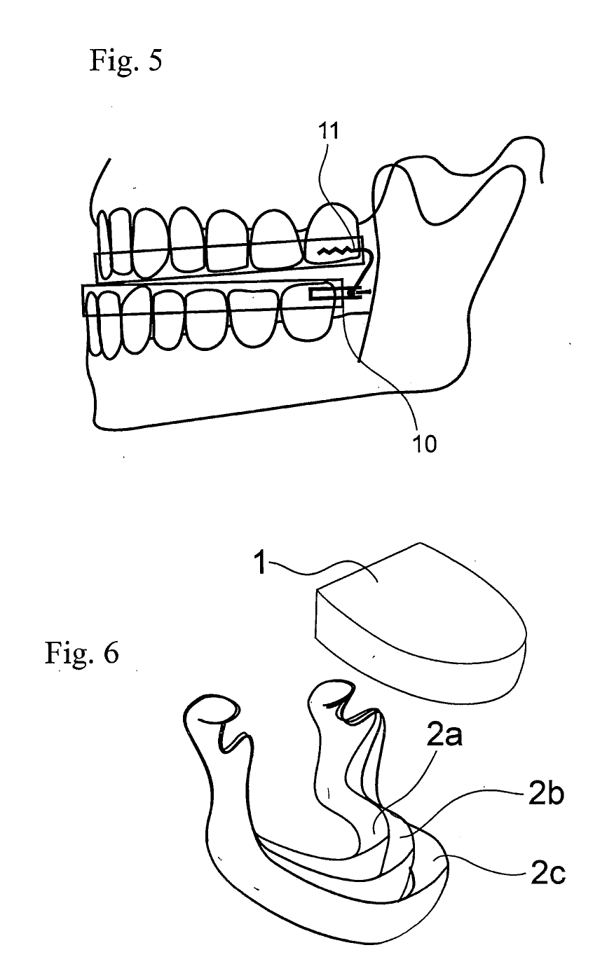

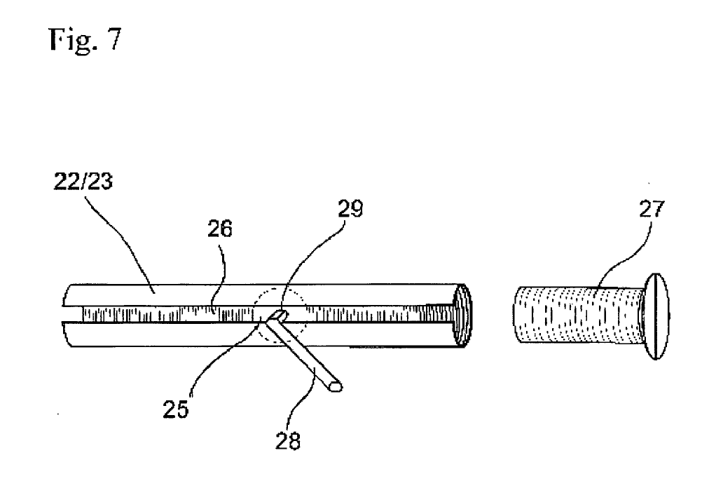

The details of the invention are described further below with the aid of the drawings FIGS. 1 and 2. Here, the drawings FIG. 1 and 2 show embodiments with elastic clamps or brackets, whilst FIGS. 3 to 7 describe the additional introduction of joints. FIGS. 4 to 6 illustrate here various arrangements of the system in side view and FIG. 6 illustrates various phases of movement of the jawbone three-dimensionally, whilst FIG. 7 illustrates the possibility of movement of the joint in FIGS. 5 and 6 in detail.

### DETAILED DESCRIPTION OF THE PREFERRED EMBODIMENTS

FIG. 1 shows the maxilla clip 1 and mandible clip 2, wherein the material over the chewing surfaces 3 is thinner by selectively more intensive heating of the deep-drawing material than on the flanks 4 to 6. The metal brackets 7 (here constructed so as to be short by way of example) can be prepared in different versions, embodied for different dentition positions and melted or polymerised into the dentition splints.

In addition, it is possible to heat the regions 10 and 11 of the brackets, after the application of markings to designate the normal position of the dentition, and to align them and cool them so that the desired forward push is achieved.

FIG. 2 shows the same arrangement but with the spirally wound metal brackets 12, making possible a further elastic region, which are to be used in particular when a further range of movement is necessary.

FIG. 3 shows the maxilla 13 with the upper row of teeth 14 and the splint 15 engaged thereon, and in addition the mandible 16 in pushed-forward position, again with row of teeth 17 and splint 18, which are connected by an elastic connection via the clamps 19 and 20. The clamps 19 and 20 are coupled here by the joint 21.

FIG. 4 shows the same arrangement with an adjustable joint 22, mounted on the end of the upper splint 15, which is illustrated in detail in FIG. 7.

FIG. 5 illustrates the same arrangement, only here the adjustable joint 23 is fastened on the lower splint 18.

FIG. 6 shows the phases of movement of these arrangements in the positions of the maxilla 1a, b and—here, however, without taking into consideration the elastic effects of the clamps 18 and 19. As can be seen, despite the protrusion, the mandible can largely carry out chewing movements in an unimpaired manner.

FIG. 7 shows the detail of an adjustable joint 22 or 23, as described in FIGS. 4 and 5. Here, a ball 25 is guided in a slotted sleeve 26 and is adjusted via the setscrew 27. An arm 28 is inserted into the ball 25, which arm is displaceable in the slot 29 of the sleeve 26 with the ball, and constitutes the connection to the splint of the respectively other row of teeth.

### LITERATURE REFERENCES

[1] American Academy of Dental Sleep Medicine ADSM, Congress Report, Philadelphia 2004, in Somnojournal March 2004
[2] An American Sleep Disorder Associations Report in SLEEP 1995, 18 (6), p. 511 ff.
[3] Hein, Rascke, Köhler, Mayer, Peter and Rühle: Leitlinie zur Diagnostik and Therapie schlafbezogener Atmungsstörungen beim Erwachsenen. Pneumaologie 2001/55, p. 339 ff.
[4] Cartwright & Samuelson: The Effects of a nonsurgical Treatment for Obstructive Sleep Apnea. JAMA, August 1982, Vol. 248, No. 6, p. 705 ff.

1. System for mandibular protrusion to prevent snoring and apnea, wherein the maxilla and mandible are held in splints and these are connected with elastic metal clamps which bring about the pushing forward of the mandible,
wherein
the metal clamps are arranged at rear ends of the splints.

2. System for mandibular protrusion to prevent snoring and apnea according to claim 1,
wherein
the splints consist of PET or similar plastic materials.

3. System for mandibular protrusion to prevent snoring and apnea according to claim 1,
wherein
the splints are connected with clamps of nickel-titanium alloys, wherein the clamps preferably have memory metal effects and are adjusted by means of heat.

4. System for mandibular protrusion to prevent snoring and apnea according to claim 1,
wherein
the clamps are kept in various shapes and embodiments and are combined with the splints respectively as required, wherein the selected clamps preferably are polymerized into the splints.

5. System for mandibular protrusion to prevent snoring and apnea according to claim 4,
wherein
the splints have cylindrical mountings into which the clamps can be fixed at a different depth.

6. System for mandibular protrusion to prevent snoring and apnea according to claim 5,
wherein
the clamps are fastened with lateral fixing screws.

7. System for mandibular protrusion to prevent snoring and apnea according to claim 7,
wherein
the clamps are fixed with coupling nuts.

8. System for mandibular protrusion to prevent snoring and apnea according to claim 4,
wherein
the clamps or their holding devices project a little from the splints, so that the splints can be easily withdrawn at these overhangs and removed from the mouth.

9. System for mandibular protrusion to prevent snoring apnea according to claim 1,
wherein
the splints are cut out in the region of the front palate to such an extent that they largely make possible unimpeded movements of the tongue and hence unimpeded speech.

10. System for mandibular protrusion to prevent snoring according to claim 1,
wherein
the clamps are made from thin but rigid material, which does not entirely cover the tooth necks.

11. System for mandibular protrusion to prevent snoring according to claim 1,
wherein
the clamps are constructed so as to be particularly thin on the masticatory surfaces, so as not to prevent the closing of the jawbones with respect to each other.

12. System for mandibular protrusion to prevent snoring according to claim 1,
wherein
if necessary, the splints have additional holding devices on and between the teeth, if particular tooth shapes (e.g. pyramid teeth) or tooth positions require this.

13. System for mandibular protrusion to prevent snoring according to claim 12,
wherein
these holding devices are clamps embracing individual teeth.

14. System for mandibular protrusion to prevent snoring according to claim 1,
wherein
the brackets or clamps which connect the splints elastically are in turn respectively connected with a joint which makes possible a movement of the jawbones in lateral and rotative direction without counter-pressure by the brackets or clamps.

15. System for mandibular protrusion to prevent snoring according to claim 14,
wherein
the joints are arranged in the interior of the mouth parallel to the axis of the temperomandibular joints.

16. System for mandibular protrusion to prevent snoring according to claim 14,
wherein
the joints are arranged respectively centrally between two bracket elements in each case.

17. System for mandibular protrusion to prevent snoring according to claim 14,
wherein
the joints are arranged at the rear ends of the upper splint.

18. System for mandibular protrusion to prevent snoring according to claim 14,
wherein
the joints are arranged at the rear ends of the lower splint.

19. System for mandibular protrusion to prevent snoring according to claim 14,
wherein
the degree of protrusion is adjustable through displacement of the bracket in a sliding sleeve by a setscrew.

20. System for mandibular protrusion to prevent snoring according to claim 14,
wherein
a joint ball is guided in a slightly inwardly inclined sleeve so that additional play is produced for a movement forwards and sideways.
